(19) United States
(12) Patent Application Publication
(10) Pub. No.: US 2021/0008526 A1
(43) Pub. Date: Jan. 14, 2021

(54) METHOD

(71) Applicant: POLYCATUK LTD., Wallingford, Oxfordshire (GB)

(72) Inventor: William PATERSON, Wallingford (GB)

(73) Assignee: PolyCatUK Ltd., Wallingford, Oxfordshire (GB)

(21) Appl. No.: 16/646,888

(22) PCT Filed: Sep. 18, 2018
(86) PCT No.: PCT/GB2018/052652

§ 371 Date: Mar. 12, 2020

(30) Foreign Application Priority Data
Sep. 22, 2017 (GB) 1715372.7

(51) Int. Cl. B01J 23/745; B01J 23/42; B01J 23/44; B01J 23/50; B01J 31/06; B01J 35/00; B01J 35/06; B01J 37/02; B01J 37/06; B01J 37/16; H01M 4/88; H01M 4/92; H01M 4/90; A61L 2/232; C23C 18/16

(52) U.S. Cl. CPC B01J 23/745; C23C 18/1658; B01J 23/44; B01J 23/50; B01J 31/06; B01J 35/0013; B01J 35/06; B01J 37/0217; B01J 37/06; B01J 37/16; H01M 4/8842; H01M 4/8817; H01M 4/925; H01M 4/9041; A61L 2/232; C23C 18/1641; C23C 18/1644; B01J 23/42

(57) ABSTRACT

The present invention relates to methods of immobilising metals on polymeric surfaces using surfactants and to products that can be formed by such methods. Polymer substrates with metal immobilised on the surface are very useful in a variety of applications. The metal is usually in the form of a nanoparticle. A major use of the invention is in catalysts. The invention can also be used in medical applications, such as to make antimicrobial surfaces.

### FIELD OF THE INVENTION

The present invention relates to methods of immobilising metals, especially in the form of metallic particles such as metallic nanoparticles, on polymeric surfaces and to products that can be formed by such methods. Polymer substrates with metal immobilised on the surface are very useful in a variety of applications. A major use of the invention is in catalysts. The invention can also be used in medical or healthcare applications, such as to make antimicrobial surfaces.

### BACKGROUND TO THE INVENTION

The present invention is concerned in the widest sense with the application of metal particles to polymers and the wide range of resulting applications, especially in catalysis.

Presently catalysts are often formed on a carbon, alumina or silica based support.

Polymers are ubiquitous, inexpensive, and can be easily moulded or formed to any required shape. Accordingly it is desirable to use a polymeric substrate as a catalyst support. However, an acceptable method for attaching metal particles directly to polymeric substrates, in particular by a simple and economic process, has not been found.

WO2007/061248 discloses catalysts for fuel cell electrodes which have a carbon based support onto which a monomer and transition metal precursor are adsorbed and then polymerised. This results in a coating of conducting polymer, to which transition metal particles are added.

US2009/0263569 discloses an electrode having an electrochemical catalyst layer which comprises a substrate with a conducting layer formed on the surface, and a conditioning layer over the conducting layer, with polymer-capped noble metal nanoclusters on the conditioning layer.

The present invention provides a method of immobilising metals on polymer surfaces, particularly complex polymers surfaces, that is simple, cost effective, and allows the metal to be deployed homogeneously in a controllable manner. Immobilising metals directly onto polymer surfaces avoids the drawbacks of the prior art methods.

### SUMMARY OF THE INVENTION

According to a first aspect the invention provides a method of immobilising metals on a polymeric substrate, the method comprising the steps of:
(1) providing a polymeric substrate that has a surface;
(2) treating the surface with an aqueous surfactant solution under conditions that lead to surfactant being partially absorbed into the surface; then
(3) adding to the surface a metal salt solution, so that ions of the metal salt become associated with partially absorbed surfactant; and
(4) adding to the metal salt solution on the surface a reducing agent, so that metal ions in the metal salt solution are reduced to metal particles.

According to a second aspect the present invention provides a polymeric substrate that has a surface with metal particles immobilised thereon by a surfactant, wherein the surfactant has a hydrophobic tail that is at least partially absorbed in the surface and a hydrophilic head that is not absorbed in the surface and to which the metal particles are attached.

According to a third aspect the present invention relates to the use of the polymeric substrate according to the second aspect of the invention as a catalyst.

The basis of this invention is the realisation that surfactants can be used to immobilise metals on substrates, particularly in the form of metal particles. This breakthrough opens the door to new catalysts, and other products in a wide variety of fields where immobilised metals are used. The process of the invention can be used to coat complex surfaces providing high surface area, particularly with metallic nanoparticles.

The method involves only a few simple steps of applying a series of solutions to the substrate, as defined above, so is synthetically easy and cost effective to carry out. The method uniquely provides in particular for the homogeneous distribution of a metal, such as a catalytic species, on a complex polymer surface, for example a microporous or sintered polymer where access to the bulk of the polymer surface by the application medium is restricted. The method allows the metal to be deployed cost effectively, being efficiently distributed on an economic substrate. The method also allows for easy control of the distribution of metal particles being immobilised, by controlling the concentration of surfactant, and for easy control of the amount of metal immobilised, by controlling the concentration of metal salt and reducing agent.

The invention also relates to polymeric substrates that have metal particles immobilised thereon by a surfactant, as can be produced by the method of the invention, and their use as catalysts. As mentioned above, in contrast to prior art products, the polymeric substrates can be complex in shape and even microporous, so could provide a very high surface area of immobilised metal particles. This is highly advantageous in many fields including catalysis.

### DESCRIPTION

The first aspect of the invention provides a method of immobilising metals on a polymeric substrate, the method comprising the first step of providing a polymeric substrate that has a surface. The substrate can be made from any polymer, but it is preferred that the polymeric substrate is hydrophobic. In a preferred embodiment the substrate is wholly or partially a polyolefin such as polyethylene or polypropylene. Polystyrenes, polyesters, polyurethanes or chlorine or fluorine containing polymers like polyvinylidene fluoride (PVDF), polyvinyl chloride (PVC), or polytetrafluoroethylene (PTFE) can also be used. Polyolefins are preferred because they are simple, cheap and ubiquitous. However, the invention could equally well be applied to other polymers and co-polymers.

The polymer is generally not a conducting polymer, as is required in some prior art processes. A conducting polymer is not necessary for the present invention, as a surfactant is used to immobilise the metal particles.

An advantage of the present invention is that the use of a series of solutions to immobilise the metals on the substrate surface means that it is particularly suited to distributing a metal on substrates with a relatively high surface area such as microporous structures. Accordingly, in a preferred embodiment the polymeric substrate is a microporous or sintered polymer where access to the bulk of the polymer surface by the application medium is restricted. By microporous we mean the substrate has multiple pores with a pore diameter of less than 5 microns, preferably less than 2 microns.

Examples of suitable complex polymers with which the invention can be used include those from the STERAPORE™ range made by Mitsubishi chemical, and the polypropylene substrates Membrane P50 or P5S made by Zena Membranes. These examples have pore sizes in the range 0.1 to 0.5 microns, which is a preferred size range for the microporous substrates of the present invention in general. In addition to complex substrates the present invention could also be applied to polymeric substrates with plain unstructured surfaces, or which are granules or fibres.

By immobilising we simply mean that the metal particles that form are attached onto the surface of the polymeric substrate. In the present invention it is thought that the metal is initially attached to the surface by the surfactant, as detailed below, and can then also become adsorbed onto the surface.

Before the second step it is important for the polymeric substrate to be clean. Accordingly, if necessary, the polymer can first be cleaned to remove any surface contamination. This would be carried out by any suitable solvent compatible with the polymer being treated, for example hexane to clean a polyolefin such as polyethylene or polypropylene.

The second step of the method of the invention is treating the surface with an aqueous surfactant solution under conditions that lead to surfactant being partly absorbed into the surface. As is well known, surfactants are molecules that have a hydrophobic portion and a hydrophilic portion. In particular, they have a hydrophobic “tail”, which is often an 8 to 18 carbon hydrocarbon, and can be aliphatic, aromatic, or a mixture of both, and a hydrophilic “head”. The hydrophilic head is typically an ionic hydrophilic head, as in ionic surfactants. That is, the head can be negatively charged as in anionic surfactants that contain anionic functional groups at their head, such as sulfates, sulfonates, phosphates, and carboxylates, positively charged as in cationic surfactants that contain cationic functional groups at their head such as quaternary ammonium salts, or zwitterionic (amphoteric) surfactants that have both cationic and anionic functional groups attached at the head.

The aqueous surfactant solution can comprise an anionic, cationic or zwitterionic surfactant. The surfactant type can be chosen depending on the ionic state of the metal ion or metal complex ion to be deposited in the metal salt solution. It is generally an anionic or cationic surfactant.

Examples of suitable anionic surfactants include dioctyl sodium sulfosuccinate, perfluorooctanesulfonate, linear alkylbenzene sulfonates, sodium lauryl ether sulfate, lignosulfonate, and sodium stearate.

In a preferred embodiment of the invention the aqueous surfactant solution comprises a cationic surfactant, preferably a cationic surfactant with a quaternary ammonium salt at the head. The inventor has found that cationic surfactants are particularly good at acting as nucleation sites for metal deposition. The cationic surfactant in the aqueous surfactant solution may comprises benzalkonium chloride (BAC), benzyl-dodecyl-dimethylammonium bromide, benzyldimethyloctadecylazanium chloride, benzylhexadecyldimethylazanium chloride or Thonzonium bromide.

A key realisation of the inventor is that a surfactant can be used to immobilise a metal, such as in the form of a metal particle, on a polymeric surface. For this to happen the polymeric substrate must be treated with the aqueous surfactant solution under conditions that lead to surfactant being partially absorbed into the surface. By partially absorbed in the surface we mean that part of the surfactant is taken into the surface by chemical or physical action. It is thought that on absorption the hydrophobic tail of the surfactant is absorbed by being entrained or dissolved, either wholly or partially, in the hydrophobic polymer surface. The hydrophilic head remains on the surface, to interact with the ions in the metal salt solution. The heat, length of contact, and concentration of surfactant in the aqueous surfactant solution are all important.

The polymer-surfactant contact needs to continue for sufficient time to ensure thorough wetting of the surface with the solution and for the absorption to take place. This will usually take at least 24 hours, often from 24, 48 or 72 hours and up to 7 days, though this will vary depending on the temperature at which the polymer can be treated without deformation or the surfactant not being degraded.

Absorption of surfactant by the polymer is accelerated by heating. The higher the temperature that is applied, the quicker the surfactant is absorbed, so temperatures up to the deformation temperature of the substrate are often used. In the case of highly processed polymers such as the microporous hollow fibres temperatures up to 60 degrees Celsius would be appropriate, whereas unprocessed polymer granules of polypropylene could be treated at autoclave temperatures 126 degrees Celsius.

The number of surfactant molecules that are partially absorbed into the polymeric surface depends on the concentration of surfactant in the aqueous solution. Obviously the higher the concentration, the more surfactant can be absorbed, and ultimately the more metal nucleation sites are provided, meaning that more metal can be immobilised, or that with the same amount of metal there will be a higher number of smaller metal particles, which can result in higher overall metal surface area and higher activity as a catalyst. Accordingly, altering the concentration of surfactant is a neat way to alter the distribution of metal on the surface.

Any excess surfactant solution can simply be rinsed off the substrate surface before the next step.

The third step of the process is the addition to the surface of a metal salt solution. The metal salt can be any metal salt, and preferably contains a multivalent metal ion.

Any metal salt where the metal is useful in catalytic, medical or other applications can be used. For catalytic applications transition metals are often used, particularly Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Lanthanum, Cerium, Hafnium, Tantalum, Tungsten, Rhenium, Osmium, Iridium, Platinum and Gold. Particularly preferred are iron, nickel, platinum, rhenium, vanadium, rhodium and silver metal salts.

Mixed metals salts can advantageously be used in the present invention.

The salts chosen should dissociate in solution to provide a metal ion that is reducible to the required metal particle. The metal ion can be simple metal ion (such as K⁺) or a metallic coordination complex (such as PtCl₆²⁻). The metal or metal complex should be a counterion capable of forming an ion pair with the surfactant immobilised on the polymer surface.

Potassium hexachloroplatinate is the preferred platinum salt. This has the following structure.

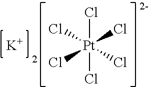

Further preferred metal salts include silver nitrate (AgNO₃), potassium hexachloropalladate(IV) (K₂PdCl₆), and sodium hexachlororhodate(III) Na₃RhCl₆.

In addition to the metal salts, the metal salt solution includes a solvent system. The solvent system usually includes water and can be all water, but is usually water mixed with a water miscible solvent to ensure efficient wetting of the polymer surface. The water miscible solvent can be any suitable solvent, such as an alcohol, acetone, dimethyl sulfoxide, or tetrahydrofuran. The solvent can be included with water at a level of between 5 and 95% of the solvent system, selected so that the solvent system is compatible with both dissolution of the metal salt and wetting of the polymer.

No special conditions are required for the third step of the process, and it can be carried out at room temperature. It is preferred that the application of the metal salt is carried out at low temperature, usually lower than room temperature (20 degrees Celsius), such as lower than 10 degrees Celsius. Lowering the temperature and therefore the rate of reaction allows for better distribution of the metal particles on the polymer surface.

Following step two of the process the hydrophilic head of the surfactant is immobilised at the surface of the substrate due to absorption of the tail portion of the surfactant. During the third step the ions in the solution become associated with partially absorbed surfactant, Without wishing to be bound by theory it is thought that the metal ion with the opposite charge forms an ion pair with the charged surfactant head. For example, for a salt such as potassium hexachloroplatinate, with a cationic surfactant the hexachloroplatinate anion will form an ion pair and with an anionic surfactant the potassium cation will form an ion pair. It seems that the ion pair can then act as a nucleation site for metal in the next step of the method.

It is preferred that the ion containing the metal to be deposited (ie the hexachloroplatinate anion in the example above) is paired with the surfactant. The surfactant can be selected according to the charge of the metal-containing ion in the metal salt solution.

Excess metal salts are provided over those that become associated with the surfactant. These remain in solution.

The fourth step of the method involves adding to the metal salt solution on the surface of the substrate a reducing agent to reduce the metal ions in solution to metal. Any reducing agent that is capable of reducing the metal ions can be used. Normally a weak reducing agent is selected. In a preferred embodiment of the invention the reducing agent is formic acid, glucose, fructose, lactose, maltose, or ascorbic acid. The reducing agent is usually added directly to the metal salt solution in contact with the polymer. Other examples of reducing agents include diisobutylaluminium hydride, sodium borohydride, hydrogen, hydrazine, and dimethyl sulfide.

The reducing agent causes the metal ions to be substantially reduced to elemental metal, i.e. having an oxidation state of 0. In other words, the charged metal ion is reduced to uncharged elemental metal. This uncharged elemental metal typically has very low or no solubility, and precipitates out of solution. As more metal precipitates, metal particles form and grow is size. The inventor has found that the precipitated metal becomes immobilised on the surface of the polymeric substrate. Without wishing to be bound by theory, it is thought that an ion pair is formed between the metal salt ion and the surfactant head, which acts as a nucleation site and an anchor for the growing metal particle. In this way the metal particle is attached to the surface through absorption of the hydrophobic tail of the surfactant. In addition it is thought that the metal particle, once formed, may be adsorbed to the surface of the polymeric substrate. It is thought that the combination of surfactant involvement (in absorption/ion pair formation/action as nucleation site) and surface adsorption immobilises the solid metal particle formed on the surface of the substrate. In a microporous polymeric substrate, the metal particles may also be physically restrained by the microporous structure.

The rate of reduction of the metal salt is controlled by a combination of both the concentration of the chosen reducing agent and the temperature of the reaction. Typically the reducing agent will be in molar excess of the metal salt. As with the previous step it is preferred that addition of reducing agent is carried out at low temperature, usually lower than room temperature (20 degrees Celsius), such as lower than 10 degrees Celsius. Lowering the temperature and therefore the rate of reaction allows for better distribution of the metal particles on the polymer surface.

The distribution of metal particles on the surface can be controlled by the surfactant concentration as set out above. The size of the metal particles is controlled by the concentration and overall level of metal salt in the solution as well as the concentration and overall level of reducing agent in the subsequently applied solution. Typically, the metal particles can have a diameter below about 1 μm, preferably below about 500 μm, more preferably below about 100 nm. The metal particles are preferably nanoparticles. By nanoparticles, we are referring to particles that have diameters in the 1-100 nm range. Typically for catalytic, medical and healthcare applications the immobilised metal particles are nanoparticles.

According to a second aspect the present invention provides a polymeric substrate that has a surface with metal particles immobilised thereon by a surfactant, wherein the surfactant has a hydrophobic tail that is at least partially absorbed in the surface and a hydrophilic head that is not absorbed in the surface and to which the metal particles are attached. This can be made by the method of the present invention, or could be accessed by a different method. As mentioned above, this product is highly advantages as the polymer substrate can be an irregular or complex shape, such as microporous. Hence the product can have a very high surface area which can be extremely beneficial in catalytic applications.

The potential uses of a product according to the invention which is a catalyst include but are not limited to any relatively low temperature reaction which could be promoted by the catalyst employed. Examples include, the conversion of carbon monoxide to carbon dioxide, the elimination of reactive oxygen species such as hydrogen peroxide and the conversion of glucose to gluconic acid. Further example include the use of platinum for the removal of VOCs (volatile organic compounds), also the removal nitrogen oxides, vanadium for the oxidation of sulphur dioxide to sulphur trioxide.

The invention can be used to make rhodium catalysts. These are used in a number of industrial processes, notably in catalytic carbonylation of methanol to produce acetic acid. It is also used to catalyze addition of hydrosilanes to molecular double bonds, a process important in manufacture of certain silicone rubbers. Rhodium catalysts are also used to reduce benzene to cyclohexane.

Further, the application of silver could both provide for catalytic use as well as use in antimicrobial applications. Other potential applications include in power, batteries and hydrogen generation.

### EXAMPLE

The metals that have been successfully applied in a method according to the invention include platinum, palladium, silver and iron. These have been applied to microporous hollow fibre, microporous sheet (in the form of ‘Celgard’ battery separator material) and ‘scree’ (staple polypropylene monofilament of approximately 5 microns diameter).

All of these applications followed the same general procedure as described above. In particular, the materials were cleaned to remove surface contamination using acetone and n-hexane. Surfactant, either benzalkonium chloride or sodium lauryl sulphate according to the nature of the metal salt in use for each metal, in 5% aqueous solution was then applied with heating at 55° C. to the base material for seven days.

The required metal salt at a concentration providing approximately 5% weight of metal with respect to the weight of the substrate was then applied to each of the substrates with mixing and this was followed by the application of a reducing agent (sodium formate) in molar excesses of approximately 4 to 10 times that of the metal salt. Mixing was continued until it became visually apparent in each case that the metal nanoparticles had formed on the various substrates used.

1. A method of immobilising metals on a polymeric substrate, the method comprising the steps of:
(1) providing a polymeric substrate that has a surface;
(2) treating the surface with an aqueous surfactant solution under conditions that lead to surfactant being partially absorbed into the surface; then
(3) adding to the surface a metal salt solution, so that ions of the metal salt become associated with partially absorbed surfactant; and
(4) adding to the metal salt solution on the surface a reducing agent, so that metal ions in the metal salt solution are reduced to metal particles.

2. A method according to claim 1, wherein the surface of the polymeric substrate is hydrophobic.

3. A method according to claim 1, wherein the polymeric substrate is a polyolefin, preferably wherein the polymeric substrate is polypropylene or polyethylene.

4. A method according to claim 1, wherein the polymeric substrate is microporous.

5. A method according to claim 1, wherein the aqueous surfactant solution comprises a cationic surfactant, preferably wherein the aqueous surfactant solution comprises benzalkonium chloride, benzyl-dodecyl-dimethylammonium bromide, benzyl dimethyloctadecylazanium chloride, benzylhexadecyldimethylazanium chloride or thonzonium bromide.

6. A method according to claim 1, wherein the metal salt solution comprises an iron, nickel, platinum, rhenium, vanadium, rhodium or silver salt, preferably wherein the metal salt solution includes potassium hexachloroplatinate.

7. A method according to claim 1, wherein the reducing agent comprises formic acid, glucose, fructose, lactose, maltose, or ascorbic acid.

8. A method according to claim 1, wherein the metal ions in the metal salt solution are reduced to metal nanoparticles, having a diameter of 1 to 100 nm.

9. A polymeric substrate that has a surface with metal particles immobilised thereon by a surfactant, wherein the surfactant has a hydrophobic tail that is at least partially absorbed in the surface and a hydrophilic head that is not absorbed in the surface and to which the metal particles are attached.

10. A polymeric substrate according to claim 9, wherein the polymeric substrate is a polyolefin, preferably wherein the polymeric substrate is polypropylene or polyethylene.

11. A polymeric substrate according to claim 9, wherein the polymeric substrate is microporous.

12. A polymeric substrate according to claim 9, wherein the surfactant comprises a cationic surfactant, preferably wherein the surfactant comprises benzalkonium chloride, benzyl-dodecyl-dimethylammonium bromide, benzyldimethyloctadecylazanium chloride, benzylhexadecyldimethylazanium chloride or thonzonium bromide.

13. A polymeric substrate according to claim 9, wherein the metal particles comprise iron, nickel, platinum, rhenium, vanadium, rhodium or silver.

14. A polymeric substrate according to claim 9, wherein the metal particles comprise metal nanoparticles having a diameter of 1 to 100 nm.

15. (canceled)
